(19) United States
(12) Patent Application Publication
(10) Pub. No.: US 2003/0088192 A1
(43) Pub. Date: May 8, 2003

(54) Test system for studying a biological fluid

(76) Inventor: Thomas Lengsfeld, Marburg (DE)

Correspondence Address: Finnegan, Henderson, Farabow,, Garrett & Dunner, L.L.P., 1301 I Street, N.W., Washington, DC, 20005-3315, US

(21) Appl. No.: 10/287,529

(22) Filed: Nov. 5, 2002

(30) Foreign Application Priority Data
Nov. 6, 2001 (DE) 101 53 963.0

(51) Int. Cl. A61B 5/00

(52) U.S. Cl. 600/584

(57) ABSTRACT

A test system for studying a biological fluid which comprises a tube- or pipe-like hollow body which is intended for receiving or passing through a biological fluid and on whose inside a support is attached on which binding molecules for one or more different antigens, antibodies, pathogens, toxins and/or exogenous or endogenous proteins to be detected in the biological fluid are arranged in fields separated from one another is described.

The invention relates to a test system for detecting small amounts of antigens, antibodies, pathogens, toxins and/or exogenous or endogenous proteins in large volumes of biological fluids.

It is known that body fluids, in particular blood, plasma and serum, are collected in medicine for diagnostic and therapeutic applications. The use of these body fluids or of the products obtained therefrom for therapeutic applications is possible only if the body fluids are free from harmful substances such as antigens, antibodies, pathogens, toxins or exogenous proteins. It is therefore necessary to study said body fluid in order to determine whether such harmful foreign substances or particular markers for such harmful foreign substances (analytes) are present. These analytes which are frequently present only in very small amounts may be in particular nucleic acids, antibodies or antigens but also viruses or prions. In addition, nonphysiological concentrations of endogenous proteins may be indicators for the presence of infective agents or diseases. Up until now, these have been detected by preparing small fractions from plasma and testing them for corresponding pathogens by means of methods known to the skilled worker, such as, for example, antigen/antibody reactions or by means of PCR. For this purpose, small-volume test amounts are, after obtaining the plasma, removed and studied. The detection methods available are limited due to the volume of material and the sensitivity of the test methods used.

The invention relates to a test method for detecting and quantifying small amounts of analytes in large volumes of liquid, in which preferably the entire amount or a substantial part of the analytes bind to support-bound specific binding molecules, are separated from the liquid and then detected or quantified either in the bound state or else after detachment from the support, using methods known per se to the skilled worker. Usually, the liquid is moved relative to the support-bound specific binding molecules, for example by gravimetric flow or by pumps.

One embodiment of the invention is a test system for studying a biological fluid, which comprises a tube- or pipe-like hollow body which is intended for receiving or passing through the biological fluid and on whose inside a support is attached on which binding molecules for one or more different antigens, antibodies, pathogens, toxins and/or exogenous or endogenous proteins to be detected in the biological fluid are arranged in fields separated from one another. The support may be flat or curved and is arranged in the test system such that it can be removed with the substances attached to the binding molecules from the hollow body to carry out further detection reactions.

It is a feature of the test system of the invention that the biological fluid to be studied can pass through the tube- or pipe-like hollow body and thus can make intensive contact with the binding molecules attached to the support.

Suitable binding molecules are all chemical organic and inorganic substances which are capable of specifically binding a particular antigen, an antibody, a pathogen, a toxin or an exogenous or endogenous protein. Polyclonal, monoclonal or recombinant antibodies or antibody fragments are very particularly suitable for this purpose.

The material of the flat support may consist of metal, glass, ceramic or a flexible material, cellulose membranes, polyamide membranes and polyester membranes having proved especially useful. Additionally, the support may be coated with substances which promote the properties of the binding molecules. A method of this kind is described by R. Jenison et al. in Clinical Chemistry 47:10, 1894-1900 (2001).

The tube- or pipe-like hollow body is generally a glass pipe or plastic pipe, a rubber tube or plastic tube or a cylinder of a hypodermic syringe.

The invention also relates to a method for detecting and/or removing antigens, antibodies, pathogens, toxins and/or exogenous or endogenous proteins in a biological fluid, in which the fluid is contacted with the tube- or pipe-like hollow body or can flow through it, and the binding molecules attached to the flat or curved support bind the substances to be detected which can then be detected either on the support in the hollow body or, preferably, after removing the support, outside the hollow body, using a specific reagent.

The substances to be detected which are attached to the binding molecules of the support can be detected on the support by using a physical method, for example the SPR (surface plasmon resonance) technique from Biacore, or preferably by means of a further enzymatic, fluorescent or radiolabeled binding molecule.

A particular advantage of the test system of the invention is the fact that the marker molecules flowing past the support provided with the binding molecules can be collected continuously over a short or long period during removal of the body fluid from the donor organism. Since large volumes of the body fluid flow past the binding molecules of the support, it is possible by using this method to detect reliably according to the invention even small amounts of markers which can otherwise be detected in a large volume of liquid only with difficulty, if at all. For example, binding molecules for viruses, prions or other pathogens, but also an antigen such as a tumor marker or another pathogen marker can be incorporated by means of a support into the pipe or tube systems required for obtaining the plasma. In this connection, those binding molecules which can capture, for example, prions or prion subtypes specifically from the body fluid passed by are selected. Suitable for passing through the body fluid are all systems through which large amounts of liquid can flow. Biological fluids which may be used are blood, plasma, serum, milk, pulmonary lavage or urine.

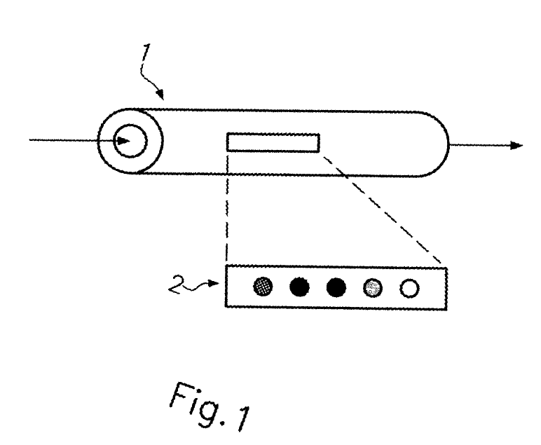

FIG. 1 shows such a flow-through system in which a flat support can be attached on which binding molecules for various antigens, antibodies, pathogens, toxins or exogenous or endogenous proteins to be detected in the body fluid are arranged in fields separated from one another. The flat support (2) attached in the pipe-like hollow body (1) can be removed from the hollow body and used for further studies. Such a test unit can be based, for example, on the protein-chip technology. For example, a prion-specific antibody can be bound on the support. The prion bound to the antibody is then detected via secondary reactions. It is also possible to cover individual detection fields first and use them only in later tests for control processes. Moreover, it is possible to detect additionally plasma reference proteins by using individual fields, which may then serve as a negative control or for a general test detection.

The invention is illustrated by the following examples.

### EXAMPLE 1

The blood removed from a donor is passed through a tube system which contains a support material in the form of a strip of 10×4 mm to which binding molecules comprising HCV antigens C22-3, C200 and NS5 have been attached for detection of hepatitis C virus. The human antibodies contained in the blood streaming past are bound by the binding molecules and then, detected.

### EXAMPLE 2

The blood taken from a donor is to be tested for HIV. For this purpose, the support is provided with a monoclonal antibody against HIV and attached in a tube through which the donor blood flows. The virus particles bound to the support are detected directly via a secondary antibody or via a virus-specific PCR reaction.

### EXAMPLE 3

Antibodies against various cytokines (BD PharMingen; (San Diego, Calif.)) from a solution of 100 μg/ml are applied in amounts of in each case 0.25 μl in fields separated from one another on a nylon membrane. 1 l of blood plasma to which cytokines at 100 μg/ml have been added are directed past the support prepared in this way. After removing the support from the hollow body and carefully washing off serum sticking to it, the cytokines attached to the binding site are detected by means of biotinylated cytokine antibodies.

1. A test system for studying a biological fluid, which comprises a tube- or pipe-like hollow body which is intended for receiving or passing through the biological fluid and on whose inside a support is attached on which binding molecules for one or more different antigens, antibodies, pathogens, toxins and/or exogenous or endogenous proteins to be detected in the biological fluid are arranged in fields separated from one another.

2. The test system as claimed in claim 1, wherein the biological fluid to be studied can pass through the tube- or pipe-like hollow body and thus contact the binding molecules fixed to the support.

3. The test system as claimed in claims 1 and 2, which comprises polyclonal, monoclonal or recombinant antibodies or antibody fragments as binding molecules.

4. The test system as claimed in claims 1 to 3, wherein the tube- or pipe-like hollow body is a glass pipe or plastic pipe, a rubber tube or plastic tube or a cylinder of a hypodermic syringe.

5. A method for detecting and/or removing antigens, antibodies, pathogens, toxins and/or exogenous or endogenous proteins from a biological fluid, wherein the fluid is contacted with the tube- or pipe-like hollow body or can flow through it and the substances to be detected which are attached to the binding molecules of the flat or curved support are then detected using a specific reagent.

6. The method as claimed in claim 5, wherein the support provided with the binding molecules is removed from the hollow body after attachment of the substances to be detected which are then detected using a physical method or by means of another enzymatic, fluorescent or radiolabeled binding molecule.

7. The method as claimed in claims 5 and 6, wherein the biological fluid employed is blood, plasma, serum, milk, pulmonary lavage or urine.

8. The method as claimed in claims 5 to 7, characterized in that the pathogen to be detected is a virus, a prion or another pathogen.

9. The method as claimed in claims 5 to 7, wherein the antigen to be detected is a tumor marker or another pathogen marker.
